(19) United States
(12) Patent Application Publication
(10) Pub. No.: US 2012/0207836 A1
(43) Pub. Date: Aug. 16, 2012

(54) DRUG DELIVERY SYSTEMS (WAFER) FOR PEDIATRIC USE

(75) Inventors: Sascha General, Berlin (DE); lldiko Terebesi, Berlin (DE); Adrian Funke, Berlin (DE)

(73) Assignee: BAYER PHARMA AKTIENGESELLSCHAFT, Berlin (DE)

(21) Appl. No.: 13/391,377

(22) PCT Filed: Aug. 19, 2010
(86) PCT No.: PCT/EP10/05083

§ 371 Date: May 1, 2012

(30) Foreign Application Priority Data
Aug. 19, 2009 (EP) 09075378.1
Aug. 20, 2009 (EP) 09075381.5

(51) Int. Cl. A61K 31/4422; A61K 9/00

(52) U.S. Cl. 424/485; 424/484; 514/356; 424/488; 424/486

(57) ABSTRACT

The present invention describes drug delivery compositions in the form of thin water-soluble films (wafers), which contain particles that comprise at least one active ingredient—which is not an estrogen and/or a progestin and/or an alkaline earth metal salt of 5-methyl-(6S)-tetrahydrofolate—and at least one protective agent. The protective agent provides effective taste-masking of the active ingredient due to limited release of the active ingredient in the mouth. The active ingredient is hence not absorbed via the buccal route, but rather via the enteral (per-oral) route. The particles contained in the wafer provided by the present invention have a particle size of below 40 μm thereby resulting in an acceptable sensation in the mouth while dissolving. Such wafers are especially suitable for pediatric use.

### FIELD OF THE INVENTION

The present invention relates to drug delivery compositions in the form of thin water-soluble films (wafers), which contain particles that comprise at least one active ingredient—which is not an estrogen and/or a progestin and/or an alkaline earth metal salt of 5-methyl-(6S)-tetrahydrofolate—and at least one protective agent. The protective agent provides effective taste-masking of the active ingredient due to limited release of the active ingredient in the mouth. The active ingredient is hence not absorbed via the buccal route, but rather via the enteral (per-oral) route. The particles contained in the wafer provided by the present invention have a particle size of below 40 μm thereby resulting in an acceptable sensation in the mouth while dissolving. Such wafers are especially suitable for pediatric use.

### BACKGROUND OF THE INVENTION

While a broad variety of medicaments (drug products) is available on the market containing many different active principles (drug substances) in many different dosage forms, these drugs are very often neither approved nor even suitable for the application to children. In consequence, pediatricians and physicians willing to treat diseases in children cannot rely on the market authorization of drug products granted by health authorities that guarantee efficacy, safety and quality of these drug products as it is usually the case in the treatment of adults.

This is partly due to the fact that the treatment of diseases in children require different dosages of drug substance than those used to treat adults. Generally speaking, the doses of a drug substance required to treat children are in most cases lower than adult doses. In many cases, the dose of a drug substance are more or less correlated to the body surface area or the body weight of a human being, so that the dose can easily be calculated. Unfortunately, this is not a generally applicable rule. In many cases, there are great differences in pharmacokinetics (i.e. absorption, distribution, metabolism and excretion) of a drug substance between children and adults. These differences can result in significant deviations from the abovementioned rule.

Another reason is that children do not typically suffer from the same disease as adults, so that they are in need of totally different drug substances.

In addition, especially young children are unable to swallow big tablets, capsules or pills. Similarly, also other dosage forms are not easy to administer to children. This holds especially true when active cooperation of a patient is required during the administration of a drug product, e.g. breathing in (nasal or pulmonal sprays) keeping still (eye drops), swallowing something (tablets etc.), and so on. On the one hand, active cooperation can often be facilitated by the insight in the necessity of a treatment beside some discomfort during the administration. This is of course difficult in young children. On the other hand, unpleasant medicines applied to children do not only reduce the willingness to cooperate during the next administration of the drug product, but sometimes even result in the opposite: active refusal of any further medication.

In order to promote the development and approval of drug products suitable for the treatment of children, the European Health Authorities request a so-called “pediatric investigation plan” to be provided by pharmaceutical companies applying for the approval of a new drug product (cf. Regulation (EC) No. 1901/2006 of European Parliament and of the Council of 12 Dec. 2006). This pediatric investigation plan shall include the development of dosage forms and clinical studies in all subsets of pediatric population (preterm newborn infants, term newborn infants, infants and toddlers, pre-school children, school children, and adolescents).

The challenges in developing pharmaceutical dosage forms for children are tremendous: the dosage forms must safeguard all quality aspects (such as dose uniformity, purity, stability etc.) and an appropriate bioavailability of the drug substance. Furthermore, the dosage form must be easy to administer to children not only by medically trained personnel, but also by their parents. Preferably, the drug product should flexibly allow for dose adaptation to e.g. the individual body weight. In addition, the excipients to be used must of course be safe and non-toxic to children. Unfortunately, not all excipients considered as safe in adults can be used equally in children, at least not in similar amounts (e.g. ethanol, propylene glycol, polyethylene glycol, several surfactants, antioxidants, and preservatives). Moreover, socio-cultural aspects have to be considered. For example in order to avoid stigmatisation, the administration of drug products to school children shall preferably happen once or twice daily at home. This sometimes calls for drug products with controlled drug substance release characteristics. If multiple applications per day are inevitable the administration should be as discrete as possible. Most importantly, the organoleptic properties must be palatable or acceptable.

These challenges and some proposals of possible solutions are very well documented in the literature, e.g.

J. Breitkreutz et al. Exp Opin Drug Deliv 4:37-45 (2007).

A Cram et al. Int J Pharm 365:1-3 (2009).

EMEA reflection paper “Formulation of choice for the paediatric population” (EMEA/CHMP/PEG/194810/2005, published 28 Jul. 2006).

The pharmaceutical industry has tried to meet this challenge by developing a number of different drug delivery systems, including rapid in-mouth disintegrating tablets, tablets which disintegrate in liquid prior to ingestion, liquids and syrups, gums, suppositories and even transdermal patches. However, each of these drug delivery systems can pose their own problems.

Transdermal patches can be inconvenient and uncomfortable as well as rather expensive to produce. Furthermore, the drug flux through the skin can also raise very complex dosing issues. Suppositories often exhibit high variations in bioavailability.

Liquids are considered particularly useful for children. However, liquids can be be relatively expensive to formulate, package and transport. Taste masking of drug substances in liquid dosage forms is a real challenge as even encapsulated drug substances can be liberated already in the dosage form by diffusion to the liquid phase. Therefore, liquid dosage forms are often provided as a taste-masked powder for reconstitution. However, while the taste masking of such liquid dosage forms is very efficient immediately after reconstitution, the unpleasant taste typically increases within the usage time of the drug product, e.g. within one to two weeks. Furthermore, parents are often unable to precisely measure the required amount of water when reconstituting the drug product. Hence, the dose accuracy of such dosage forms is more than questionable.

Tablets that can be dissolved in a liquid before ingestion can also be useful. However, they can also be quite inconvenient in that they require liquid and a drinking container to be provided. Furthermore, time is required for disintegration and/or dissolution, even when effervescent tablets are used. Finally, these drug delivery systems can be quite messy as they typically leave a particulate and/or scum in the glass. Rapid in-mouth disintegrating tablets, such as chewable or self disintegrating tablets offer great convenience. However, chewable or self-disintegrating tablets often present real taste masking problems as the act of chewing can disrupt protective coatings. Furthermore, chewable or self-disintegrating tablets are often associated with an unpleasant mouthfeel. Moreover, the fear of swallowing, chewing, or choking on such solid shaped articles is still a concern in certain populations. In addition, the fragility/friability of such porous, and low-pressure moulded tablets makes them difficult to carry, store, handle and administer to patients, especially the children and the elderly.

Developing a drug product which has an acceptable sensation in the mouth while dissolving is a major challenge. Therefore texture is very important, as well as taste. Texture is determined by a number of factors: graininess and viscosity and hardness and stickiness. Beside this, the changes of these mechanical properties during mastication are decisive for the acceptability of the sensation in the mouth. It is known from the literature (J. Prescott et al., Cross-cultural comparisons of Japanese and Australian responses to manipulation of sweetness in foods, Food Quality and Preference, Vol. 8, Issue 1, 1997, 45-55) that there are cultural differences in acceptable or pleasant sensations in the mouths. The strength of jaw muscles and the emergence and the number of teeth also play an important role, especially in the elderly and in children of all ages. A baby with no teeth and weak jaw muscles has a different sense of texture than an adult. For this reason baby food is usually semi-solid. Danisco, a manufacturer of drinking yoghurt, has tested the acceptability of texture including graininess of its products. The results (Tracy M. Mosteller, Drinkable Yogurts and Smoothies, Danisco USA Inc.) reveal that even casein particles as small as 40-60 μm were perceived as “grainy” and unpleasant.

Another relevant investigation of texture, particles size and graininess threshold in the mouth showed that chewing sensation is different for different materials (E. Imai, K. Saito et al., Effect of Physical Properties of Food Particles on the Degree of Graininess perceived in the Mouth; Journal of Texture Studies 30, 1999, 59-88). These differences in the sensation threshold depend on grain hardness, form and changes during mastication. If the grains adsorb water easily or if they dissolve in saliva the sensation threshold is often higher than for grains that maintain the mechanical properties. For a selection of grains the threshold was found to lie between 23 μms for cellulose and 50 μms Casein. These are the examples showing the lowest sensation threshold of all grains tested. Convincingly these results correlate with the Danisco tests for drinking yoghurt.

Therefore grains of a size of 40-60 μm or above which do not change their mechanical properties during mastication are perceptible in the mouth.

Any encapsulation process for taste masking must lead to grains that do not change their properties during mastication.

It can not be determined conclusively whether children like or dislike graininess. In order to ensure safe application of medication to children it is important to remain below the sensation threshold. This is especially the case for those without teeth or strong jaw muscles as this influences sensory perception.

### OBJECT OF THE INVENTION

Consequently the task is to create a reliable delivery systems with improved compliance, i.e. where dosage is easy and allows for a discrete administration wherever and whenever needed. Any unpleasant taste of the drug substance should be effectively masked, and the application should not appear grainy as it is applied.

Thus, there is a need for reliable delivery systems with improved compliance and the drug delivery should exhibit a palatable mouthfeel, i.e. the application should not appear grainy as it is applied Furthermore, the drug delivery should allow for a dose adaptation to the individual patient.

Such delivery systems should be especially suitable for pediatric use, i.e. for use in adolescents in the age group of up to 18 years (0 to 18 years).

In summary, there is a need for drug delivery systems where the unpleasant taste of the active ingredient is effectively masked. In addition, or alternatively, there is a need for a drug delivery system which is bioequivalent to a standard IR oral tablet or capsule, but which, at the same time, do not possess the drawbacks of such a standard oral IR tablet or capsule.

### SUMMARY OF THE INVENTION

The present inventor has provided a drug delivery system which, on the one hand, takes advantage of the attractive properties of wafers, but which, one the other hand, ensures that the unpleasant taste of the active ingredient(s) is effectively masked. This has been achieved by ensuring that once the wafer matrix is (quickly) dissolved in the saliva the active ingredient is, due to the presence of an appropriate protective agent, not dissolved in the mouth (and hence not administered via the buccal route), but is rather, by normal deglutition, transported to the stomach and/or the intestine where the active ingredient is effectively released. The drug delivery system of the invention is flexible in the sense that it may easily be adapted to a system which is bioequivalent to a standard IR oral tablet or capsule reference product.

Chewable taste-masked pharmaceutical compositions are described in U.S. Pat. No. 4,800,087.

Taste-masked orally disintegrating tablets (ODTs) are described in US 2006/0105038.

Taste-masking coating systems are described in WO 00/30617.

Taste-masked wafers are described in WO 03/030883.

Taste-masked powders and granules are described in EP 1 787 640.

Medicament-containing particles and solid preparations containing the particles are described in US 2007/0148230.

Non-mucoadhesive film dosage forms and techniques and methodologies for retarding the absorption of drugs from orally disintegrating films through the oral mucosa are described in WO 2008/040534. According to this document, mixing of donepezil with Eudragit® EPO results in immediate release characteristics of the active compound.

Solid dosage forms containing an edible alkaline agent as taste masking agent are described in WO 2007/109057.

Compositions and methods for mucosal delivery are described in WO 00/42992. This document further discloses dosage units wherein the active agent is encapsulated within a polymer.

Taste-masked pharmaceutical compositions prepared by coacervation are described in WO 2006/055142.

Compositions comprising sustained-release particles are described in U.S. Pat. No. 7,255,876.

WO 2007/074472 teaches that filler particles, e.g. having a particle size of >100 μm, give a coarse, gritty or sandy mouth feel when ingested as a mouth-dissolving tablet. Furthermore, this document discloses means to improve the mouth feel.

Xu et al., Int J Pharm 2008;359;63 describe taste masking microspeheres for orally disintegrating tablets. However, the active agent is released relatively fast from these particles and complete taste masking is not achieved.

US 2007/0292479 describes film-shaped systems for transmucosal buccal application. Furthermore, the film-shaped systems described in US 2007/0292479 contain high amounts of cyclodextrin.

S I Pather, M J Rathbone and S Senel, Expert Opin Drug Deliv 2008;5;531 review the current status and the future of buccal drug delivery systems and provide an insight into the difficulties and challenges in developing buccal dosage forms.

In the light of these prior art documents, the problems to be solved by the present invention include, but are not limited, to
formulate taste masked particles in such a size that they fit into drug delivery systems in the form of thin films (wafers);
formulate taste masked particles in such a way that they do not give any coarse, gritty or sandy mouth feel when released from the drug delivery systems into the mouth
uniformly incorporate taste masked particles into unit dosage forms in the form of thin films (wafers)
incorporate taste masked particles into thin water-soluble films comprising a water-soluble matrix polymer without dissolving or extracting said taste masked particles during manufacturing and/or storage

In a first aspect, the present invention relates to a unit dosage form comprising a thin water-soluble film matrix, wherein
a) said film matrix comprises at least one water-soluble matrix polymer;
b) said film matrix comprises particles where said particles comprise at least one active ingredient and at least one protective agent, and where said particles have a d₉₀ particle size of 40 μm; and
c) said film matrix has a thickness of ≦300 μm,
   with the provisio that the active ingredient is not an estrogen and/or a progestin and/or an alkaline earth metal salt of 5-methyl-(6S)-tetrahydrofolate.

A grain size of below 40 μm allows for safe application for children. Thereby it is assured that the application does not appear grainy as the dosage form is applied.

Unit dosage forms of this type comprising a progestin or a progestin and an estrogen are already described in PCT/EP2009/060298 which are not within the scope of the present invention and unit dosage forms of this type comprising an alkaline earth metal salt of 5-methyl-(6S)-tetrahydrofolate alone or together with a progestin and/or an estrogen are already described in EP 09167733.6 which are not within the scope of this invention. Other aspects of the present invention will be apparent from the below description and the appended claims.

### DETAILED DESCRIPTION OF THE INVENTION

The term “active ingredient” according to the invention is intended to mean any of a variety of pharmaceutical actives, medicaments and bioactive substances with the provisio that active ingredient does not mean an estrogen and/or a progestin.

Examples of basic drugs as an “active ingredient” include, but are not limited to, levobetaxolol hydrochloride, roxithromycin, dicyclomine hydrochloride, montelukast sodium, dextromethorphan hydrobromide, diphenhydramine hydrochloride, orbifloxacin, ciprofloxacin, enoxacin, grepafloxacin, levofloxacin, lomefloxacin, nalidixic acid, acycloguanosine, tinidazole, deferiprone, cimetidine. oxycodone, remacemide, nicotine, morphine, hydrocodone, rivastigmine, propanolol, betaxolol, chlorpheniramine, and paroxetine.

Examples of acidic drugs as an “active ingredient” include, but are not limited to, nicotinic acid, mefanamic acid, indomethacin, diclofenac, repaglinide, ketoprofen, ibuprofen, valproic acid, lansoprazole, ambroxol, omeprazole, acetaminophen, topiramate, amphotericin B, and carbemazepime.

In addititon to the drugs provided specifically above any of a variety of pharmaceutical actives, medicaments and bioactive active substances may be used in forming the complexates. The following is a non-exhaustive list of exemplary actives.

Examples of useful drugs include ace-inhibitors, antianginal drugs, anti-arrhythmias, anti-asthmatics, anti-cholesterolemics, analgesics, anesthetics, anticonvulsants, anti-depressants, anti-diabetic agents, anti-diarrhea preparations, antidotes, anti-histamines, anti-hypertensive drags, anti-inflammatory agents, anti-lipid agents, anti-manics, anti-nauseants, anti-stroke agents, anti-thyroid preparations, anti-tumor drugs, anti-viral agents, acne drags, alkaloids, amino acid preparations, anti-tussives, anti-uricemic drugs, anti-viral drags, anabolic preparations, systemic and non-systemic anti-infective agents, antineoplastics, antiparkinsonian agents, anti-rheumatic agents, appetite stimulants, biological response modifiers, blood modifiers, bone metabolism regulators, cardiovascular agents, central nervous system stimulates, cholinesterase inhibitors, contraceptives, decongestants, dietary supplements, dopamine receptor agonists, endometriosis management agents, enzymes, erectile dysfunction therapies, fertility agents, gastrointestinal agents, homeopathic remedies, hormones, hypercalcemia and hypocalcemia management agents, immunomodulators, immunosuppressives, migraine preparations, motion sickness treatments, muscle relaxants, obesity management agents, osteoporosis preparations, oxytocics, parasympatholytics, parasympathomimetics, prostaglandins, psychotherapeutic agents, respiratory agents, sedatives, smoking cessation aids, sympatholytics, tremor preparations, urinary tract agents, vasodilators, laxatives, antacids, ion exchange resins, anti-pyretics, appetite suppressants, expectorants, anti-anxiety agents, anti-ulcer agents, anti-inflammatory substances, coronary dilators, cerebral dilators, peripheral vasodilators, psycho-tropics, stimulants, anti-hypertensive drugs, vasoconstrictors, migraine treatments, antibiotics, tranquilizers, anti-psychotics, anti-tumor drugs, anti-coagulants, anti-thrombotic drugs, hypnotics, anti-emetics, anti-nauseants, anti-convulsants, neuromuscular drugs, hyper- and hypo-glycemic agents, thyroid and anti-thyroid preparations, diuretics, anti-spasmodics, terine relaxants, anti-obesity drugs, erythropoietic drugs, anti-asthmatics, cough suppressants, mucolytics, DNA and genetic modifying drugs, and combinations thereof. Examples of medicating active ingredients contemplated for use in the present invention include antacids, H₂-antagonists, and analgesics. For example, antacid dosages can be prepared using the ingredients calcium carbonate alone or in combination with magnesium hydroxide, and/or aluminum hydroxide. Moreover, antacids can be used in combination with H2-antagonists.

Analgesics include opiates and opiate derivatives, such as oxycodone, ibuprofen, aspirin, acetaminophen, and combinations thereof that may optionally include caffeine.

Other preferred drugs for other preferred active ingredients for use in the present invention include anti-diarrheals such as immodium AD, anti-histamines, anti-tussives, decongestants, vitamins, and breath fresheners. Common drugs used alone or in combination for colds, pain, fever, cough, congestion, runny nose and allergies, such as acetaminophen, chlorpheniramine maleate, dextromethorphan, pseudoephedrine HCl and diphenhydramine may be included in the film compositions of the present invention.

Also contemplated for use herein are anxiolytics such as alprazolam; anti-psychotics such as clozopin and haloperidol; non-steroidal anti-inflammatories (NSAID's) such as dicyclofenacs and etodolac, anti-histamines such as loratadine, astemizole, nabumetone, and Clemastine; anti-emetics such as granisetron hydrochloride and nabilone; bronchodilators such as Bentolin®, albuterol sulfate; antidepressants such as fluoxetine hydrochloride, sertraline hydrochloride, and paroxtine hydrochloride; anti-migraines such as Imigra®, ACE-inhibitors such as enalaprilat, captopril and lisinopril; anti-Alzheimer's agents, such as nicergoline; and Ca-antagonists such as nifedipine, and verapamil hydrochloride.

The popular H2-antagonists which are contemplated for use in the present invention include cimetidine, ranitidine hydrochloride, famotidine, nizatidien, ebrotidine, mifentidine, roxatidine, pisatidine and aceroxatidine.

Active antacid ingredients include, but are not limited to, the following: aluminum hydroxide, dihydroxyaluminum aminoacetate, aminoacetic acid, aluminum phosphate, dihydroxyaluminum sodium carbonate, bicarbonate, bismuth aluminate, bismuth carbonate, bismuth subcarbonate, bismuth subgallate, bismuth subnitrate, bismuth subsilysiiate, calcium carbonate, calcium phosphate, citrate ion (acid or salt), amino acetic acid, hydrate magnesium aluminate sulfate, magaldrate, magnesium aluminosilicate, magnesium carbonate, magnesium glycinate, magnesium hydroxide, magnesium oxide, magnesium trisilicate, milk solids, aluminum mono-ordibasic calcium phosphate, iricalcium phosphate, potassium 0 bicarbonate, sodium tartrate, sodium bicarbonate, magnesium aluminosilicates, tartaric acids and salts.

The active ingredient may be comprised in the particles in its free form or may be comprised in form of a pharmaceuticaly acceptable salt, solvate or derivative thereof, such as in the form of an ether, ester or a complex thereof, e.g. a cyclodextrin complex.

The term “cyclodextrin complex” or “active ingredient complexed with cyclodextrin” is intended to mean a complex between an active ingredient and a cyclodextrin, wherein the active ingredient molecule is at least partially inserted into the cavity of a cyclodextrin molecule. The molar ratio between the active ingredient and the cyclodextrin may be adjusted to any desirable value. In interesting embodiments of the invention, a molar ratio between the active ingredient and the cyclodextrin is from about 2:1 to 1:10, preferably from about 1:1 to 1:5, most preferably from about 1:1 to 1:3, such as 1:1 or 1:2. Furthermore, the active ingredient molecule may at least partially be inserted into the cavity of two or more cyclodextrin molecules, e.g. a single active ingredient molecule may be inserted into two cyclodextrin molecules to give 1:2 ratio between active ingredient and cyclodextrin. Similarly, the complex may contain more than one active ingredient molecule at least partially inserted into a single cyclodextrin molecule, e.g. two active ingredient molecules may be at least partially inserted into a single cyclodextrin molecule to give a 2:1 ratio between active ingredient and cyclodextrin. Complexes between an active ingredient and cyclodextrins may be obtained by methods known in the art.

The term “cyclodextrin” is intended to mean a cyclodextrin or a derivative thereof as well as mixtures of various cyclodextrins, mixtures of various derivatives of cyclodextrins and mixtures of various cyclodextrins and their derivatives. The cyclodextrin may be selected from the group consisting of α-cyclodextrin, β-cyclodextrin, γ-cyclodextrin and derivatives thereof. The cyclodextrin may be modified such that some or all of the primary or secondary hydroxyl groups of the macrocycle are alkylated or acylated. Methods of modifying these hydroxyl groups are well known to the person skilled in the art and many such modified cyclodextrins are commercially available. Thus, some or all of the hydroxyl groups of the cyclodextrin may have been substituted with an O—R group or an O—C(O)—R group, wherein R is an optionally substituted C₁₋₆-alkyl, an optionally substituted C₂₋₆-alkenyl, an optionally substituted C₂₋₆-alkynyl, an optionally substituted aryl or heteroaryl group. Thus, R may be a methyl, an ethyl, a propyl, a butyl, a pentyl, or a hexyl group, i.e. O—C(O)—R may be an acetate. Furthermore, the hydroxyl groups may be per-benzylated, per-benzoylated, benzylated or benzoylated on just one face of the macrocycle, i.e. only 1, 2, 3, 4, 5 or 6 hydroxyl groups is/are benzylated or benzoylated. Naturally, the hydroxyl groups may also be per-alkylated or per-acylated, such as per-methylated or per-acetylated, alkylated or acylated, such as methylated or acetylated, on just one face of the macrocycle, i.e. only 1, 2, 3, 4, 5 or 6 hydroxyl groups is/are alkylated or acylated, such as methylated or acetylated. Commonly used cyclodextrins are hydroxypropyl-β-cyclodextrin, DIMEB, RAMEB and sulfoalkyl ether cyclodextrins, such as sulfobutyl ether cyclodextrin (available under the trademark Captisol®). Although cyclodextrin-complexed active ingredients are indeed contemplated, the composition, in one embodiment of the invention, does not contain any cyclodextrin.

In the present context, the term “C₁₋₆-alkyl” is intended to mean a linear or branched saturated hydrocarbon chain having from one to six carbon atoms, such as methyl; ethyl; propyl, such as n-propyl and isopropyl; butyl, such as n-butyl, isobutyl, sec-butyl and tert-butyl; pentyl, such as n-pentyl, isopentyl and neopentyl; and hexyl, such as n-hexyl and isohexyl. Likewise, the term “C₁₋₄-alkyl” is intended to mean a linear or branched saturated hydrocarbon chain having from one to four carbon atoms, such as methyl; ethyl; propyl, such as n-propyl and isopropyl; and butyl, such as n-butyl, isobutyl, sec-butyl and tert-butyl.

In a preferred embodiment, the unit dosage form of the invention does not contain a cyclodextrin.

As indicated above, the particles containing the active ingredient should be prepared in such a way that as little active ingredient as possible is released in the mouth, while as much active ingredient as possible is released in the stomach or, optionally, in the small intestine. This can be achieved by combining the active ingredient with a protective agent as will be discussed infra.

This aforementioned embodiment is especially required if the active ingredient has an unpleasant, for instance bitter taste (in the mouth) and/or if the active ingredient has to be protected, for instance because it is instable and prone to degradation if not protected.

In case the active ingredient has not to be protected it can be present in the matrix of the dosage unit in dispersed, preferably molecularly dispersed form or in amorphous form or in form of small crystals.

As will be known by the person skilled in the art, the typical residence time of disintegrating dosage forms in the mouth is typically below 3 minutes. In case (micro)particles are released from such dosage forms in the mouth, the same applies to these (micro)particles. Thus, the typical residence time of these (micro)particles in the mouth is about 3 minutes (this is meant to include the time from intake until the disintegration of the dosage form). Consequently, effective taste-masking may be investigated by in vitro dissolution tests in small volumes of a liquid simulating the saliva, and it can reasonably be assumed that effective taste-masking is achieved when, in the early time points from 0 to 3 minutes, the drug substance in 10 ml of a dissolution medium (typically an aqueous solution of pH 6) is either not detected or the detected amount is below the threshold for identifying its taste. It is evident that the absolute threshold for identifying the taste of a drug substance is dependent on the nature and dose of the drug substance.

Thus, in order to effectively mask the unpleasant taste of the active ingredient, the protective agent must ensure that no or only very limited amounts of the active ingredient is dissolved under conditions simulating the conditions prevailing in the mouth. More particularly, it is preferred that less than 25% (w/w), such as less than 20% (w/w), more preferably less than 15% (w/w), such as less than 10% (w/w), most preferably less than 5% (w/w) of the active ingredient is dissolved from the unit dosage form within 3 minutes as determined in an in vitro dissolution experiment representing the conditions in the mouth. Basically, the dosage form is placed onto the bottom of a glass beaker. Then, 10 ml of simulated saliva pH 6.0 (composition: 1.436 g disodium phosphate dihydrate, 7.98 g monopotassium phosphate, and 8.0 g sodium chloride are dissolved in 950 ml water, adjusted to pH 6.0 and made up to 1000 ml) at 37° C. as dissolution medium is added into the beaker. Typically, the experiment is performed without any stirring or shaking (except for a gentle shaking within the first five seconds of the experiment in order to safeguard complete wetting of the dosage form), provided that the dosage form is formulated in such a way that it disintegrates completely within 3 minutes applying this procedure. If the dosage form is not formulated in such a way, stirring or shaking may be applied in a way that ensures complete disintegration of the dosage form within 3 minutes. After 3 minutes, the content of the beaker is inspected visually, and a sample of the liquid is drawn, filtered and analyzed for the content of the drug substance.

In order to investigate and assess the taste-masking properties of the protected particles before incorporation in the unit dosage form of the invention, the dissolution test described in Xu et al., Int J Pharm 2008;359;63 may be applied. In a preferred embodiment of the invention less than 20% (w/w), more preferably less than 15% (w/w), most preferably less than 10% (w/w) of the active ingredient is dissolved from the protected particles within 5 minutes as determined by a dissolution apparatus type II using distilled water at 37° C. as the dissolution media and 100 rpm as the stirring rate.

As indicated above, it is of utmost importance that the active ingredient is quickly and effectively released in the stomach and/or the intestine. As will be understood by the skilled person also this effect may be simulated by in vitro dissolution tests, and it can reasonably be assumed that effective release of the active ingredient in the stomach and/or the intestine is achieved if at least 70% (w/w), more preferably at least 80% (w/w), most preferably at least 90% (w/w) of the active ingredient is dissolved from the unit dosage form within 30 minutes as determined by United States Pharmacopoeia (USP) XXXI Paddle Method (apparatus 2) using 900-1000 ml of a suitable dissolution medium at 37° C. and 50-100 rpm, preferably either 50, 75 or 100 rpm, as the stirring rate. Alternatively, the unit dosage form may be assayed for a shorter period of time under similar conditions. In such cases, it is preferred that at least 70% (w/w), more preferably at least 80% (w/w), most preferably at least 90% (w/w) of the active ingredient is dissolved from the unit dosage form within 20 minutes, more preferably within 15 minutes, as determined by USP XXXI Paddle Method (apparatus 2) using 900-1000 ml a suitable dissolution medium at 37° C. as the dissolution media and 50-100 rpm, preferably either 50, 75 or 100 rpm, as the stirring rate.

The suitable dissolution medium may be selected so that it reflects physiological conditions in the stomach and/or the intestine and specific properties of the unit dosage form. Thus, a suitable dissolution medium may be selected from e.g. water, aqueous buffer solutions of pH 1-8 (such as pH 1.0, 1.2, 1.3, 2.0, 4.5, 6.0 and 6.8), aqueous buffer solutions of pH 1-8 (such as pH 1.0, 1.2, 1.3, 2.0, 4.5, 6.0 and 6.8) with the addition of 0.1-3% (w/v) sodium dodecyl sulphate, simulated gastric fluid, simulated intestinal fluid (fasted or fed state).

Examples of simulated gastric fluids and simulated intestinal fluids are described in the USP XXXI. There are, however, other compositions of simulated body fluids known in the pharmaceutical literature. As mentioned supra, the exact composition of the dissolution medium should be selected in such a way that it reflects the physiological conditions in the stomach and/or the intestine and the specific properties, for instance the solubility of the active ingredient of the unit dosage form.

A variety of materials, which are all well-known to the person skilled in the art, can be employed as the protective agent according to the present invention. Specific examples of such protective agents include cationic polymethacrylates and waxes.

In a preferred embodiment of the invention, the protective agent is a cationic polymethacrylate copolymer based on di-C₁₋₄-alkyl-amino-C₁₋₄-alkyl methacrylates and neutral methacrylic acid C₁₋₆-alkyl esters. In a more preferred embodiment of the invention, the cationic polymethacrylate is a copolymer based on dimethylaminoethyl methacrylate and neutral methacrylic acid C₁₋₄-alkyl esters, such as a copolymer based on dimethyl-aminoethyl methacrylate, methacrylic acid methyl ester and methacrylic acid butyl ester. A particular preferred cationic polymethacrylate is poly(butyl methacrylate, (2-dimethyl aminoethyl) methacrylate, methyl methacrylate) 1:2:1. The cationic polymethacrylates mentioned above typically have an average molecular mass in the range of from 100,000 to 500,000 Da, such as an average molecular mass in the range of from 100,000 to 300,000 Da, e.g. an average molecular mass in the range of from 100,000 to 250,000 Da, preferably an average molecular mass in the range of from 100,000 to 200,000 such as an average molecular mass in the range of from 125,000 to 175,000 Da, e.g. an average molecular mass of about 150,000 Da.

Such cationic polymethacrylates are available from Degussa, Germany, under the trade name Eudragit® E. In particular Eudragit® E 100 is preferred.

In another preferred embodiment of the invention, the protective agent is a wax. Examples of waxes include animal waxes, such as beewax, chinese wax, shellac wax, spermaceti wax and wool wax; vegetable waxes, such as carnauba wax, bayberry wax, candelilla wax, castor wax, esparto wax, ouricury wax, rice bran wax and soy wax; mineral waxes, such as ceresin wax, montan wax, ozocerite wax and peat wax; petroleum waxes, such as paraffin wax and microcrystalline wax; and synthetic waxes, such as polyethylene waxes, Fischer-Tropsch waxes, esterified and/or saponified waxes, substituted amide waxes and polymerised α-olefines. A particular preferred wax is carnauba wax.

The weight ratio between the progestin and the wax is typically in the range of from 1:1 to 1:4, such as about 1:1, about 1:2, about 1:3 or about 1:4.

As discussed above, the particles comprising the active ingredient and the protective agent should release as little active ingredient as possible in the mouth, while as much active ingredient as possible should be dissolved in the stomach and/or the intestine. This can be achieved, e.g., by embedding the active ingredient in the protective agent, for example in such a way that the active ingredient is present in a solid dispersion in the protective agent. This embodiment is particularly preferred when the protective agent is a cationic polymethacrylate.

Alternatively, the active ingredient may be coated with the protective agent. This embodiment is particularly preferred when the protective agent is a wax.

In the present context, the term “solid dispersion” is used in its commonly accepted meaning, i.e. as a dispersion, wherein the dispersed phase consists of amorphous particles or crystalline particles or individual molecules (molecular dispersion). Thus, when used herein, the term “solid dispersion” means any solid system in which a component A (the active ingredient) is dispersed at a level of small particles or even at the molecular level (molecular dispersion) within another component B (such as a protective agent).

In the present context, the term “molecularly dispersed” or “molecular dispersion” is used in its commonly accepted meaning, i.e. as a dispersion, wherein the dispersed phase consists of individual molecules. Thus, when used herein, the term “molecularly dispersed” or “molecular dispersion” means any solid, semi-solid or liquid system in which a component A (an actice ingredient) is dispersed at the molecular level within another component B (such as a protective agent), so that component A neither can be detected in crystalline form by X-ray diffraction analysis, nor be detected in particulate form, by any microscopic technique. It should also be understood that component A is dissolved in component B regardless of the nature and physical state of B. Thus, the term “molecularly dispersed” may be used interchangeably with the term “molecularly dissolved”.

As can be seen from the examples provided herein, the particle size of the particles comprising the active ingredient and the protecting agent is, at least to a certain extent, dependent on the applied protective agent. When carnauba wax is used as the protective agent, the d₉₀ particle size measurement leads in some cases to unplausible high values which may be attributed to the formation of secondary aggregates and agglomerates. Such aggregates and agglomerates are easily separated during the manufacturing of the wafers. The particle size values specified below refer to the primary particles and not to the particle size of aggregates and agglomerates. As indicated above, the particles comprising the active ingredient and the protective agent have a d₉₀ particle size of ≦40 μm, and a d₅₀ particle size of ≦15 μm. When used herein, the term “d₉₀ particle size” is intended to mean that the particle size distribution is so that at least 90% of the particles have a particle diameter of less than the specified value, calculated from the volume distribution curve under the presumption of spherical particles. In a similar way, the term “d₅₀ particle size” is intended to mean that the particle size distribution is so that at least 50% of the particles have a particle diameter of less than the specified value, calculated from the volume distribution curve under the presumption of spherical particles.

Therefore, it is important to note that whenever the terms “particle size”, “particle size distribution”, “particle diameter”, “d₉₀”, “d₅₀”, etc., are used herein it should be understood that the specific values or ranges used in connection therewith are always meant to be determined from the volume distribution curve under the presumption of spherical particles. The particle size distribution may be determined by various techniques, e.g. laser diffraction, and will be known to the person skilled in the art. The particles may be spherical, substantially spherical, or non-spherical, such as irregularly shaped particles or ellipsoidally shaped particles. Ellipsoidally shaped particles or ellipsoids are desirable because of their ability to maintain uniformity in the film forming matrix as they tend to settle to a lesser degree as compared to spherical particles. The particle size distribution of the particles comprising the active ingredient and the protective agent, when incorporated in the wafer, may be determined by dissolving the film forming matrix, separation of the protected particles, and drying the protected particles. The particle size distribution of the resulting particles may be determined as described above, e.g. by laser diffraction. For example, a Sympatec Helos laser diffractometer with a Sympatec Rhodos module aerial dispersion system can be used. (Focal length 125 mm, volume of airstream 2.5 m³/h, prepressure 2 bar, dispersion pressure 3-4 bar, optical concentration 0.8-20%, measurement time: 2 seconds, optical model: Fraunhofer under the assumption of spherical particles).

Concerning the particles comprising the active ingredient and the protective agent, these particles typically constitute less than 60% by weight of the unit dosage form, preferably less than 50% by weight of the unit dosage form, more preferably less than 40% by weight of the unit dosage form. As will be understood, the amount of particles comprising the active ingredient and the protective agent is dependent on the potency of the selected active ingredient. Accordingly, the particles comprising the active ingredient and the protective agent generally constitute 0.1-50% by weight of the unit dosage form, preferably 1-40%, such as 2-40%, e.g. 5-30% by weight of the unit dosage form. Specific values include about 12%, about 15%, about 20%, and about 30% by weight of the unit dosage form.

As will be understood the particles comprising the active ingredient(s) and the protective agent may contain additional excipients. However, in a preferred embodiment of the invention the particles consist essentially of the active ingredient(s) and the protective agent.

As will be understood from the examples provided herein, the encapsulation efficiency is high and typically above 80%, such as above 85%, e.g. above 90%. Thus, the encapsulation efficiency is typically in the range of from 80-100%, such as in the range of from 85-100%, e.g. in the range of from 90-100%. When used herein, the term “encapsulation efficiency” means the ratio of the amount of active ingredient incorporated in the protected particles versus the amount of active ingredient used for manufacturing of the protected particles.

The term “water-soluble film matrix”, when used herein, refers to a thin film which comprises, or consists of, a water-soluble polymer, particles comprising at least one active ingredient and at least one protective agent, and optionally other auxiliary components dissolved or dispersed in the water-soluble polymer.

As used herein, the term “water-soluble polymer” refers to a polymer that is at least partially soluble in water, and preferably fully or predominantly soluble in water, or absorbs water. Polymers that absorb water are often referred to as being “water-swellable polymers”. The materials useful for the present invention may be water-soluble or water-swellable at room temperature (about 20° C.) and other temperatures, such as temperatures exceeding room temperature. Moreover, the materials may be water-soluble or water-swellable at pressures less than atmospheric pressure. Desirably, the water-soluble polymers are water-soluble, or water-swellable having at least 20% by weight water uptake. Water-swellable polymers having 25% by weight, or more, water uptake, are also useful.

The unit dosage forms of the present invention formed from such water-soluble polymers are desirably sufficiently water-soluble to be dissolvable upon contact with bodily fluids, in particular saliva.

The water-soluble matrix polymer (typically constituting the major part of the water-soluble film matrix) can be selected from the group consisting of a cellulosic material, a synthetic polymer, a gum, a protein, a starch, a glucan and mixtures thereof.

Examples of cellulosic materials suitable for the purposes described herein include carboxymethyl cellulose, methyl cellulose, ethyl cellulose, hydroxymethyl cellulose, hydroxyethyl cellulose, hydroxypropyl cellulose, hydroxymethylpropyl cellulose, hydroxypropylmethyl cellulose and combinations thereof. Particularly preferred cellulosic materials are hydroxypropylmethyl cellulose and hydroxypropyl cellulose, in particular hydroxypropylmethyl cellulose.

Examples of synthetic polymers include polymers commonly used as immediate-release (IR) coatings for pharmaceuticals, such as the polyvinyl alcohol polyethylene glycol (PVA-PEG) copolymers, which are commercially available in different grades under the trademark Kollicoat® IR. Further examples of synthetic polymers include polyacrylic acid and polyacrylic acid derivatives. For steroids which are unsubstituted in the 6- and/or 7-position it was observed that the above-mentioned synthetic polymers, in particular a PVA-PEG copolymer, provide a stabilising effect on the active substances present in the unit dosage form by limiting the oxidative degradation of the active substance(s) which are unsubstituted in the 6- and/or 7-position. This advantageous stabilising effect by the synthetic polymer, in particular a PVA-PEG copolymer, will probably occur in other active ingredients, too. This effect is particularly pronounced when the active agent is dispersed, in particular molecularly dispersed, in the film matrix. Such degradations are well known in the field and is typical a problem in connection with the shelf life of the final solid preparation (see, for example, T. Hurley et al. Steroids 2002;67;165-174 and Van D. Reif et al. Pharmaceutical Research 1987;4;54-58).

Examples of water-soluble gums include gum arable, xanthan gum, tragacanth, acacia, carageenan, guar gum, locust bean gum, pectin, alginates and combinations thereof.

Useful water-soluble protein polymers include gelatine, zein, gluten, soy protein, soy protein isolate, whey protein, whey protein isolate, casein, levin, collagen and combinations thereof.

Examples of useful starches include gelatinised, modified or unmodified starches. The source of the starches may vary and include pullulan, tapioca, rice, corn, potato, wheat and combinations thereof.

Additional water-soluble polymers, which may be used in accordance with the present invention, include dextrin, dextran and combinations thereof, as well as chitin, chitosin and combinations thereof, polydextrose and fructose oligomers.

The amount of active ingredient incorporated in the unit dosage form of the invention is, of course, also dependent on the potency of the selected active ingredient, but will generally be in the range of from 0.1-30% (w/w) calculated on the basis of the unit dosage form. Typically, the amount of active ingredient incorporated in the unit dosage form of the invention is 0.5-25% (w/w), such as 1-20% (w/w), preferably 1-15% (w/w), such as 2-10% (w/w), e.g. about 6% (w/w) or about 7.5% (w/w).

The amount (dosage) of the active ingredient in the unit dosage form has to be adopted for pediatric use depending on the nature of the active ingredient. Normally the daily amount needed and to be administered to children is lower than the amount which has to be administered per day to an adult person. In some cases it may also be required to administer higher daily doses to children than to adults, for instance in case of higher metaboliv turnover of an active ingredient in children.

In addition to the water-soluble matrix polymer and the particles comprising the active ingredient and the protective agent, the unit dosage form of the invention may include a variety of various auxiliary components, such as taste-masking agents; organoleptic agents, such as sweeteners, taste modifiers and flavours, anti- and de-foaming agents; plasticizing agents; surfactants; emulsifying agents; agents improving the wetting of the particles; thickening agents; binding agents; cooling agents; saliva-stimulating agents, such as menthol; antimicrobial agents; colorants; etc. In a preferred embodiment of the invention, the unit dosage form does not contain an absorption enhancer.

Suitable sweeteners include both natural and artificial sweeteners. Specific examples of suitable sweeteners include, e.g.:
a) water-soluble sweetening agents such as sugar alcohols, monosaccharides, disaccharides and polysaccharides such as maltit, xylit, mannit, sorbit, xylose, ribose, glucose (dextrose), mannose, galactose, fructose (levulose), sucrose (sugar), maltose, invert sugar (a mixture of fructose and glucose derived from sucrose), partially hydrolyzed starch, corn syrup solids, dihydrochalcones, monellin, steviosides, and glycyrrhizin;
b) water-soluble artificial sweeteners such as the soluble saccharin salts, i.e., sodium or calcium saccharin salts, cyclamate salts, the sodium, ammonium or calcium salt of 3,4-dihydro-6-methyl-1,2,3-oxathiazine-4-one-2,2-dioxide, the potassium salt of 3,4-dihydro-6-methyl-1,2,3-oxathiazine-4-one-2,2-dioxide (acesulfame-K), the free acid form of saccharin, and the like;
c) dipeptide-based sweeteners, such as L-aspartic acid derived sweeteners, such as L-aspartyl-L-phenylalanine methyl ester (aspartame), L-alpha-aspartyl-N-(2,2,4,4 5 tetramethyl-3-thietanyl)-D-alaninamide hydrate, methyl esters of L-aspartyl-L phenylglycerin and L-aspartyl-L-2,5, dihydrophenylglycine, L-aspartyl-2,5-dihydro-L phenylalanine, L-aspartyl-L-(1-cyclohexyen)-alanine, and the like;
d) water-soluble sweeteners derived from naturally occurring water-soluble sweeteners, such as a chlorinated derivatives of ordinary sugar (sucrose), known, for example, under the product description of sucralose®; and
e) protein-based sweeteners such as thaurnatoccous danielli (Thaurnatin I and II).

In general, an effective amount of sweetener is utilised to provide the level of sweetness desired for a particular unit dosage form, and this amount will vary with the sweetener selected. This amount will normally be from about 0.01% to about 20% by weight, preferably from about 0.05% to about 10% by weight, of the unit dosage form. These amounts may be used to achieve a desired level of sweetness independent from the flavour level achieved from any optional flavour oils used.

Useful flavours (or flavouring agents) include natural and artificial flavours. These flavourings may be chosen from synthetic flavour oils and flavouring aromatics, and/or oils, oleo resins and extracts derived from plants, leaves, flowers, fruits and so forth, and combinations thereof. Non-limiting examples of flavour oils include: spearmint oil, cinnamon oil, peppermint oil, clove oil, bay oil, thyme oil, cedar leaf oil, oil of nutmeg, oil of sage, and oil of bitter almonds. Also useful are artificial, natural or synthetic fruit flavours such as vanilla, chocolate, coffee, cocoa and citrus oil, including lemon, orange, grape, lime and grapefruit, and fruit essences including apple, pear, peach, strawberry, raspberry, cherry, plum, pineapple, apricot and the like. These flavourings can be used individually or in combination. Commonly used flavours include mints such as peppermint, artificial vanilla, cinnamon derivatives, and various fruit flavours, whether employed individually or in combination. Flavourings such as aldehydes and esters including cinnamylacetate, cinnamaldehyde, citral, diethylacetal, dihydrocarvyl acetate, eugenyl formate, p-methylanisole, and the like may also be used. Further examples of aldehyde flavourings include, but are not limited to acetaldehyde (apple); benzaldehyde (cherry, almond); cinnamicaldehyde (cinnamon); citral, i.e., alpha citral (lemon, lime); neral, i.e. beta citral (lemon, lime); decanal (orange, lemon); ethyl vanillin (vanilla, cream); heliotropine, i.e., piperonal (vanilla, cream); vanillin (vanilla, cream); alpha-amyl cinnamaldehyde (spicy fruity flavours); butyraldehyde (butter, cheese); valeraldehyde (butter, cheese); citronellal (modified, many types); decanal (citrus fruits); aldehyde C-8 (citrus fruits); aldehyde C-9 (citrus fruits); aldehyde C-12 (citrus fruits); 2-ethyl butyraldehyde (berry fruits); hexenal, i.e. trans-2 (berry fruits); tolyl aldehyde (cherry, almond); veratraldehyde (vanilla); 12,6-dimethyl-5-heptenal, i.e. melonal (melon); 2-dimethyloctanal (greenfruit); and 2-dodecenal (citrus, mandarin); cherry; grape; essential oils, like menthol; mixtures thereof; and the like.

The amount of flavouring employed is normally a matter of preference, subject to such factors as flavour type, individual flavour, and strength desired. The amount may be varied in order to obtain the result desired in the final product. Such variations are within the capabilities of those skilled in the art without the need for undue experimentation. In general, amounts from about 0.01% to about 10% by weight of the film matrix are employed.

As discussed above, the unit dosage form may also include one or more surfactants, one or more emulsifying agents and/or other agents which aid in improving the wetting of the particles.

Examples of surfactants include nonionic, anionic, cationic and amphoteric surfactants. In particular, nonionic surfactants are preferred.

Examples of nonionic surfactants include, but are not limited to, the following:
Reaction products of a natural or hydrogenated castor oil and ethylene oxide. The natural or hydrogenated castor oil may be reacted with ethylene oxide in a molar ratio of from about 1:35 to about 1:60, with optional removal of the PEG component from the products. The PEG-hydrogenated castor oils, available under the trademark Cremophor®, are especially suitable, in particular Cremophor® S9 (polyoxyethylene-400-monostearate) and Cremophor® EL (polyoxyl 35 castor oil).
Polyoxyethylene sorbitan fatty acid esters, also known as polysorbates, e.g., mono- and tri-lauryl, palmityl, stearyl and oleyl esters of the type known and commercially available under the trademark Tween®, including the following products:
   Tween® 20 [polyoxyethylene(20)sorbitanmonolaurate]
   Tween® 40 [polyoxyethylene(20)sorbitanmonopalmitate]
   Tween® 60 [polyoxyethylene(20)sorbitanmonostearate]
   Tween® 65 [polyoxyethylene(20)sorbitantristearate]
   Tween® 80 [polyoxyethylene(20)sorbitanmonooleate]
   Tween® 81 [polyoxyethylene(5)sorbitanmonooleate]
   Tween® 85 [polyoxyethylene(20)sorbitantrioleate]

Although PEG itself does not function as a surfactant, a variety of PEG-fatty acid esters have useful surfactant properties. Among the PEG-fatty acid monoesters, esters of lauric acid, oleic acid and stearic acid are most useful.
Sorbitan fatty acid esters, also known as spans, such as sorbitan monolaurate (span 20), sorbitan monostearate (span 60) and sorbitan monooleate (span 80).
Polyoxyethylene fatty acid esters, e.g., polyoxyethylene stearic acid esters of the type known and commercially available under the trademark Myrj®.
Polyoxyethylene-polyoxypropylene co-polymers and block co-polymers, e.g., of the type known and commercially available under the trademark Pluronic®, Emkalyx® and Poloxamer®
Dioctylsulfosuccinate or di-[2-ethylhexyl]-succinate.
Phospholipids, in particular, lecithins. Suitable lecithins include, in particular, soybean lecithins.
PEG mono- and di-fatty acid esters, such as PEG dicaprylate, also known and commercially available under the trademark Miglyol® 840, PEG dilaurate, PEG hydroxystearate, PEG isostearate, PEG laurate, PEG ricinoleate, and PEG stearate.
Polyoxyethylene alkyl ethers, such as those commercially available under the trademark Brij®, e.g., Brij® 92V and Brij® 35.
Fatty acid monoglycerides, e.g., glycerol monostearate and glycerol monolaurate.
Saccharose fatty acid esters.
Cyclodextrins.
Tocopherol esters, e.g., tocopheryl acetate and tocopheryl acid succinate.
Succinate esters, e.g., dioctylsulfosuccinate or related compounds, such as di-[2-ethylhexyl]-succinate.

Examples of anionic surfactants include, but are not limited to, sulfosuccinates, phosphates, sulfates and sulfonates. Specific examples of anionic surfactants are sodium lauryl sulfate, ammonium lauryl sulfate, ammonium stearate, alpha olefin sulfonate, ammonium laureth sulfate, ammonium laureth ether sulfate, ammonium stearate, sodium laureth sulfate, sodium octyl sulfate, sodium sulfonate, sodium sulfosuccinimate, sodium tridecyl ether sulfate and triethanolamine lauryl sulfate.

The amount may be varied in order to obtain the result desired in the final product. Such variations are within the capabilities of those skilled in the art without the need for undue experimentation. In general, amounts from about 0.01% to about 10% by weight of the film matrix are employed, preferably from about 0.05% to 5% by weight of the film matrix are employed.

As discussed above, the unit dosage form may also include an anti-foaming and/or de-foaming agent, such as simethicone, which is a combination of a polymethylsiloxane and silicon dioxide. Simethicone acts as either an anti-foaming or de-foaming agent which reduces or eliminates air from the film composition. Anti-foaming agents will aid in preventing the introduction of air into the composition, while de-foaming agents will aid removing air from the composition.

The unit dosage form of the invention is most preferably in the form of a thin film, which dissolves fast mainly due to the large surface area of the film, which wets quickly when exposed to the moist oral environment. Contrary to fast-dissolving tablets, which are usually soft, friable and/or brittle, the film is solid and strong, but still flexible and does not require special packaging. As indicated above, the film is thin and can be carried in the patient's pocket, wallet or pocket book.

The film may be applied under or on the tongue, to the upper palatine, to the inner cheeks or any oral mucosal tissue, of the female mammal. The film may be rectangular, oval, circular, or, if desired, a specific shape, cut to the shape of the tongue, the palatine or the inner cheeks, may be applied. The film is rapidly hydrated and will adhere onto the site of application where it then rapidly disintegrates.

Concerning the dimensions of the unit dosage form of the invention, the water-soluble film forming matrix is formed into a dry film which has a thickness of ≦300 μm, preferably ≦250 μm, more preferably ≦200 μm, most preferably ≦150 μm, such as ≦120 μm, e.g. ≦100 μm. As will be understood from the discussion above concerning the particle size of the particles comprising the progestin and the protective agent, the particle size, and therefore also to a certain extent the thickness of the film matrix, is somewhat dependent on the actually chosen protective agent. It is generally preferred, however, that the thickness of the film matrix is in the range of from 10-150 μm, such as 20-125 μm, e.g. 30-100 μm. More preferably, the thickness of the film matrix is in the range of from 35-90 μm, in particular in the range of from 40-80 μm. Specific, and preferred, examples include thicknesses of about 30 μm, about 40 μm, about 50 μm, about 60 μm, about 70 μm, about 80 μm, about 90 μm, about 100 μm, about 110 μm or about 120 μm.

The surface dimension (surface area) of the film matrix is typically in the range of from 2-8 cm², such as in the range of from 3-8 cm², e.g. in the range of from 4-7 cm², more preferably in the range of from 4-6 cm². Specific, and preferred, examples of the surface area include surface areas of about 3, 3.5, 4, 4.5, 5, 5.5 or 6 cm². Most preferably, the surface area is about 4, 4.5, 5 or 5.5 cm².

The total weight of the film matrix will typically be in the range of from 5-200 mg, such as in the range of from 5-150 mg, e.g. in the range of from 10-100 mg. More preferably, the total weight of the film matrix is in the range of from 10-75 mg, such as in the range of from 10-50 mg. Specific, and preferred, examples of the weight of the film matrix include weights of about 15 mg, about 20 mg, about 25 mg, about 30 mg, about 35 mg, about 40 mg, about 45 mg or about 50 mg.

The unit dosage form may be prepared and adhered to a second layer, i.e. a support or backing layer (liner) from which it is removed prior to use, i.e. before being introduced into the oral cavity. Preferably, the support or backing material is not water-soluble and may preferably consist of polyethylene-terephthalate, or other suitable materials well known to the skilled person.

In one embodiment of the invention, the unit dosage form may contain at least one further active ingredient which—like the first active ingredient termed before as the active ingredient—is incorporated in the unit dosage form in a way allowing the further active ingredient not to be absorbed via the buccal route, i.e. so that as little estrogen as possible is dissolved in the mouth, while as much further active ingredient as possible is dissolved in the stomach and/or the intestine. This may be achieved by combining the further active ingredient with a protective agent in a similar way as discussed supra in connection with the first active ingredient.

### Manufacture

The unit dosage form of the invention may be prepared by processes and methods as shown in the examples and as described in WO 2007/073911.

The protected particles are typically prepared by dissolving the protective agent in a suitable organic solvent after which the active ingredient is added. Depending on the selection of the protective agent, the protective agent is either deposited on the surface of active ingredient particles (e.g. in the case carnauba wax is used as protective agent), or the active ingredient is incorporated as solid dispersion into particles comprising the protective agent and the active ingredient (e.g. in the case a cationic polymethacrylate copolymer is used as protective agent).

After removal of the organic solvent the resulting microparticles are dried and optionally milled and sieved. The milling equipment is selected according to the properties of the particles and the desired particle size, e.g. rotor mills or air jet mills may be used. For the milling process it might be necessary to cool the mill feed, e.g. with dry ice addition to the feed. Alternatively, the active ingredient may be dissolved together with the protective agent and spray-dried at a suitable temperature, e.g. 30-50° C., e.g. at a temperature of about 35° C. Typically, the protected particles prepared by spray-drying had a d₅₀ particle size of about 5-15 μm.

The matrix polymer solution (coating solution) is typically prepared by adding the water-soluble matrix polymer to a suitable solvent, such as water or a mixture of an alcohol and water. As mentioned supra, it may be preferred in some cases that the protected particles, if the protective agent is a wax (in particular carnauba wax) that a surfactant is added. As will be understood, the time and conditions needed to dissolve the water-soluble matrix polymer will depend on the polymer and the solvent used. Thus, in some cases the water-soluble matrix polymer may dissolve easily at room temperature and with only gentle stirring, while in other cases it will be necessary to apply heat and vigorous stirring to the system. In a typical embodiment, the mixture is stirred for 1-4 hours, preferably for about 2 hours, or until a solution is obtained. The solution is typically stirred at a temperature of 60-80° C., such as about 70° C. After cooling to room temperature, the protected particles are optionally dispersed in a small volume of solvent or solvent mixtures and then poured into the matrix polymer solution and mixed thoroughly. The final mixing step and the optional pre-dispersing step as well can be performed by any method known to the skilled person, e.g. by using a pestle and mortar, or by stirring with an appropriate stirrer, such as a propeller stirrer, or by high sheer mixing, or by using rotor-stator mixing devices, such as ultra-turrax, and/or applying ultrasound. Important thereby is the viscosity of the matrix solution that must hinder the particles from sedimentation during the following processes and at the same time must guarantee a homogenous distribution of the particles. The viscosity is dependent of polymer in solution, the solvents used, and the particle or grain size. The resulting solution (coating solution) can be used for coating immediately or within a few days, preferably within one day. The various amounts of solvent, matrix polymer, etc. are adjusted to reach a solid content of the coating solution of about 5-50% by weight, preferably 10-40% by weight, in particular 20-40% by weight, such as about 25% by weight, about 30% by weight, about 33% by weight, about 35% by weight and about 40% by weight.

Other excipients, auxiliary components and/or active drug substances may be added during any of the above mentioned steps.

As discussed supra the unit dosage form of the invention may contain a second active ingredient, which may be dispersed, preferably molecularly dispersed, in the water-soluble film matrix. In this case, the further (second) active ingredient is dissolved in a suitable solvent, such as ethanol and/or propylene glycol. This solution can be added to the solvents used for the coating solution before addition of the water-soluble matrix polymer. Alternatively, the solution can also be added after the water-soluble matrix polymer is already dissolved. In this case, the solution can be added either before, together or after the addition of the protected particles, before the final mixing step is performed.

If needed, the coating solution is degassed before being spread out on a suitable support or backing layer (liner). Examples of suitable liners include polyethylene-terephthalate (PET) liners, such as Perlasic® LF75 (available from Perlen Converting), Loparex® LF2000 (available from Loparex BV) and Scotchpack® 9742 (available from 3M Drug delivery Systems). In one embodiment of the invention, the coating solution is spread out with the aid of a spreading box onto a suitable liner and dried for 12-24 hours at room temperature. A thin opaque film is then produced, which is subsequently cut or punched into pieces of the desired size and shape. Alternatively, the coating solution is coated as a thin film onto a suitable liner and in-line dried using an automated coating and drying equipment (e.g. by Coatema Coating Machinery GmbH, Dormagen, Germany) using a drying temperature of 40-100° C. A thin opaque film is then produced, which is subsequently cut or punched into pieces of the desired size and shape.

The units can be adjusted to specific dosages by adjusting the height, the area, are the content of the compound and may then be administered to warm-blooded animals, incl. humans.

### Further Embodiments

1. A unit dosage form comprising a thin water-soluble film matrix, wherein
a) said film matrix comprises at least one water-soluble matrix polymer;
b) said film matrix comprises particles where said particles comprise at least one active ingredient and at least one protective agent, and where said particles have a d₉₀ particle size of ≦40 μm; and
c) said film matrix has a thickness of ≦300 μm,
   with the provisio that the active ingredient is not an estrogen and/or a progestin and/or an alkaline earth metal salt of 5-methyl-(6S)-tetrahydrofolate

2. The unit dosage form according to embodiment 1, wherein said active ingredient is embedded in said protective agent.

3. The unit dosage form according to embodiment 2, wherein said active ingredient is present in a solid dispersion in said protective agent.

4. The unit dosage form according to embodiment 1, wherein said active ingredient is coated with said protective agent.

5. The unit dosage form according to any of the preceding embodiments, wherein said protective agent is a cationic polymethacrylate.

6. The unit dosage form according to embodiment 5, wherein said cationic polymethacrylate is a copolymer based on di-C₁₋₄-alkyl-amino-C₁₋₄-alkyl methacrylates and neutral methacrylic acid C₁₋₆-alkyl esters.

7. The unit dosage form according to embodiment 6, wherein said cationic polymethacrylate is a copolymer based on dimethylaminoethyl methacrylate and neutral methacrylic acid C₁₋₄-alkyl esters.

8. The unit dosage form according to embodiment 7, wherein said cationic polymethacrylate is a copolymer based on dimethyl-aminoethyl methacrylate, methacrylic acid methyl ester and methacrylic acid butyl ester.

9. The unit dosage form according to embodiment 8, wherein said cationic polymethacrylate is poly(butyl methacrylate, (2-dimethyl aminoethyl) methacrylate, methyl methacrylate) 1:2:1.

10. The unit dosage form according to any of embodiments 1-4, wherein said protective agent is a wax.

11. The unit dosage form according to embodiment 10, wherein said wax is carnauba wax.

12. The unit dosage form according to any of the preceding embodiments, wherein said water-soluble matrix polymer is selected from the group consisting of a cellulosic material, a gum, a protein, a starch, a synthetic polymer, a glucan, and mixtures thereof.

13. The unit dosage form according to embodiment 12, wherein said water-soluble matrix polymer is a cellulosic material.

14. The unit dosage form according to embodiment 13, wherein said cellulosic material is selected from the group consisting of carboxymethyl cellulose, methyl cellulose, ethyl cellulose, hydroxymethyl cellulose, hydroxyethyl cellulose, hydroxypropyl cellulose, hydroxymethylpropyl cellulose and hydroxypropylmethyl cellulose.

15. The unit dosage form according to embodiment 14, wherein said cellulosic material is hydroxypropylmethyl cellulose or hydroxypropyl cellulose, preferably hydroxypropylmethyl cellulose.

16. The unit dosage form according to embodiment 12, wherein said water-soluble matrix polymer is a synthetic polymer.

17. The unit dosage form according to embodiment 16, wherein said synthetic polymer is a polyvinyl alcohol polyethylene glycol (PVA-PEG) copolymer.

18. The unit dosage form according to any of the preceding embodiments, wherein said film matrix has a thickness of ≦250 μm, preferably ≦200 μm, such as ≦150 μm, more preferably ≦120, such as ≦100 μm.

19. The unit dosage form according to embodiment 18, wherein said film matrix has a thickness in the range of from 10-150 μm, such as 20-125 μm, e.g. 30-100 μm, preferably 35-90 μm, more preferably 40-80 μm.

20. The unit dosage form according to any of the preceding embodiments, wherein said unit dosage form further comprises at least one further active ingredient.

21. The unit dosage form according to any of the preceding embodiments, wherein said unit dosage form comprises at least one surfactant.

22. The unit dosage form according to any of the preceding embodiments, wherein said film matrix comprises at least one surfactant.

23. The unit dosage form according to any of the preceding embodiments, wherein less than 25% (w/w), preferably less than 20% (w/w), more preferably less than 15% (w/w), most preferably less than 5% (w/w) of the active ingredient is dissolved from the unit dosage form within 3 minutes when the unit dosage form is placed into a beaker with 10 ml of simulated saliva pH 6.0 at 37° C. as dissolution medium.

24. The unit dosage form according to any of the preceding embodiments for pediatric use as a medicament.

The invention is further illustrated by the following non-limiting examples.

### Examples

### Example 1

### Preparation of Particles Comprising a Protective Agent

### Example 1A: Nifedipin/Eudragit

1 gram of Nifedipine is dissolved in 50 ml of Acetone. 19 g Eudragit E 100 is added to this solution and subsequently dissolved with stirring of the solution. A table stirrer at mean velocity and elevated temperature (35° C.) is used. The 50 ml solution is then casted into Teflon-coated aluminium foil formed into a cup-like shape. The solution in the cup is put into a laminar flow box for 48 h at room temperature to remove the solvent. A clear crystal free, solid block consisting of 95% Eudragit E100 and 5% Nifedipin [w/w] is obtained. The block is broken into pieces of an area of about 1-3 cm². These pieces are milled in an air mill LSM 50 stainless steel with the following parameters adjusted; injector nozzle d=1.1 mm; diffuser d=3.8 to 5.7 mm; milling nozzle d=0.7 mm; outlet 9.7 mm, at 5 bar air pressure and a feed of 2.15 g/min. The milling is done two times. The obtained particles have a diameter d₅₀ of 11 μm, determined with a Helos (H0710) and Rodos with standard parameters adjusted. This powder of particles is the starting material for further processes.

The particle size distribution obtained after milling twice as described in Example 1A is d₅₀ about 11 μm, d₉₀ about 25 μm and d₉₉ about 35 μm.

### Example 1B: Ethinylestradiol/Carnauba Wax (As Illustrative Example)

80 g of carnauba wax (Pharm. Grade) was dissolved in 1 kg of n-heptane at 60° C. in a 2 litre double-walled glass beaker while stirred at 400 rpm until a clear solution was obtained.

80 g of micronized (d₅₀=1.5 μm; d₉₀=4.0 μm) ethinylestradiol was added slowly to the solution to avoid clumping while the stirring rate was adjusted to 600 rpm. The mixture was cooled to 20° C. at a cooling rate of 20° C./hour to yield the drug containing microparticles coated with Carnauba wax.

The ethinylestradiol-containing microparticles were filtrated using a cellulose acetate filter membrane and a glass filter unit. The microparticles were subsequently washed with 300 ml ethanol (96%) to remove n-heptane residues and non-encapsulated ethinylestradiol.

The filtered microparticles were transferred to a glass bowl and dried for 2 hours at 30° C.

The resulting particles had the following particle size distribution:

| d₅₀ (μm) | d₇₀ (μm) | d₉₀ (μm) |
|---|---|---|
| 11.5 | 18 | 36 |

The encapsulation efficiency was greater than 90%.

### Example 1C: Ethinylestradiol/Eudragit® E 100 (As Illustrative Example for Spray-Drying)

10 g of ethinylestradiol and 90 g of Eudragit® E 100 were dissolved in 1000 ml of ethanol (96%) and spray-dried with a laboratory spray drier (Büchi 190, Switzerland). The ethinylestradiol was found to be molecularly dispersed in a solid dispersion in the protective agent, as confirmed by X-ray analysis. The resulting protected particles, wherein the ethinylestradiol is present in molecularly dispersed form in the protective agent, had a d₅₀ particle size of 5.5 μm and a d₉₀ particle size of 13.8 μm. The protected particles are stored protected from heat (e.g. in a refrigerator) until further use. The encapsulation efficiency was greater than 90%.

### Example 2

### Preparation of Particle-Containing Film Matrix (Coating) Solutions

### Example 2A: Nifedipin Coating Solution

36 g purified water is heated to 60° C. and 8 g hydroxy-propyl cellulose (Klucel EF) are added and dissolved after cooling. A clear polymer solution is obtained. 6 g of the powder obtained in Example 1A were placed in a beaker and the polymer solution was added stepwise. The particles were homogenously dispersed using a pistil. The obtained dispersion is the coating solution.

### Example 2B: Nifedipin Coating Solution

32.5 g of purified water is heated to 60° C. and 8 g polyvinyl acetate-polyethylene glycol-copolymer (Kollicoat IR) are added. The polymer is dissolved after cooling to obtain a transparent polymer solution. 8 g of the particles obtained in Example 1A are placed in a beaker and the polymer solution is adedded stepwise. The particles are distributed homogenously using a pistil to obtain the coating solution.

### Example 3

### Preparation of Wafers

### Example 3A: Nifedipin Wafer

The coating solution obtained in Example 2A is coated to a film using a 800 μm scraper. The film obtained is dried at room temperature. The obtained laminate is used to punch single units, so called wafers.

### Example 3B: Nifedipin Wafer

The coating solution obtained in Example 2B is coated to a film using a 800 μm scraper. The obtained film is dried at room temperature. The obtained laminate is used to punch single units, so called wafers.

### Example 3C

The coating solution is degassed and coated as a thin film onto a polyethylene-terephthalate (PET) liner (Perlasic® LF75) and in-line dried using an automated coating and drying equipment (Coatema Coating Machinery GmbH, Dormagen, Germany). A drying temperature of 70° C. is applied. An opaque film with a thickness of about 70 μm is produced. Wafers with a total weight of about 35 mg are obtained by punching out samples of 5 cm² size.

### Example 4

### Pharmaceutical Drug Product

The film matrix contains the active ingredient homogeneously distributed such, that the surface area of the film correlates to the amount of active in a linear manner.

To achieve the possibility of a flexible dose adaptation to the individual patient, the surface of the film matrix is consisting of at least once the size, but mostly a multiple of the size required for one dosage to be administered.

The required dose to by applied for each patient is defined in dependence of the age, height, weight, gender or other defined physiological parameter and provided to the user together with the product.

The user identifies the required dose by determining the surface area of the film product containing the required dose according to the information provided.

Then, the user separates the required surface area of the film from the remaining film matrix right before administration.

To secure a precise dosing during the separation of the required surface area of the film two embodiments are provided according to the invention:
(1) Pre-defined separation marks (e.g. by tear-off perforation) to facilitate to accurately separate the required surface area of the film matrix
(2) In-situ definition and separation of the required surface area of the film matrix.

### Examples for Embodiment (1)

### Example 4A

Single wafer with with pre-defined separation marks for separation in several parts, e.g. for separation in 4 parts according FIG. 1.

### Example 4B

A wafer stripe with pre-defined separation marks, from which one or several area parts can be separated at once (FIG. 2).

Packaging of the wafer stripe may be similar to those also used in the food industry, such as for chewing gums. One example is presented in FIG. 3.

Other technical solutions may be possible, such as e.g. used and established in the market for adhesive stripes.
The separation marks required to accurately separate the required surface area of the film matrix may be prepared e.g. by perforation, pre-cutting or pre-punching with remaining small contact points or any other technical solution established and known by those, skilled in the art.

### Example for Embodiment (2)

The technical solution for the in-situ definition and separation of the required surface area of the film requires, that a technical solution is provided together with the film matrix, e.g. a technical device, which assists the precise separation of the required surface area.
Technical solutions may be derived e.g. from the example in FIG. 3, as depicted in FIG. 4, e.g. by introducing a scale bar on the surface of the packaging, which allows a metering of the wafer stripe length according to the required dose. The correlation of the dose to the wafer stripe length can be provided with the packaging leaflet or also printed onto the outer surface of the packaging.
Alternatively the technical device may include an additional mechanism inserted into the packaging, which allows a definition of the required size upfront before actuation of the device. Such technical solutions are already established in the market e.g. for the application of pre-defined amounts of liquids, as used for example in insulin pens.
Such devices can optionally have also a mechanism for presentation of the film product after separation of the required area from the wafer stripe to facilitate the removal of the wafer by the user for immediate administration. Such technical solutions are known and established in the market e.g. for commercially available adhesives stripes, too.

Therefore the present invention also relates to a pharmaceutical drug product comprising a thin water-soluble film-matrix, wherein
a) said film-matrix comprises a water-soluble polymer and at least one pharmaceutically active compound (active ingredient)
b) said pharmaceutically active compound is distributed homogeneously within the matrix so that the amount of pharmaceutically active compound is directly and linearly correlated with the area of the matrix and
c) said pharmaceutical drug product is provided in a manner which allows for separation of discrete portions (unit dosage forms) of the pharmaceutical drug product (metering and adjusting the dose according to the area of the separated portion).

1. A unit dosage form comprising a thin water-soluble film matrix, wherein
a) said film matrix comprises at least one water-soluble matrix polymer;
b) said film matrix comprises particles where said particles comprise at least one active ingredient and at least one protective agent, and where said particles have a d₉₀ particle size of ≦40 μm; and
c) said film matrix has a thickness of ≦300 μm,
with the provisio that the active ingredient is not an estrogen and/or a progestin and/or an alkaline earth metal salt of 5-methyl-(6S)-tetrahydrofolate.

2. The unit dosage form according to claim 1, wherein said active ingredient is embedded in said protective agent.

3. The unit dosage form according to claim 2, wherein said active ingredient is present in a solid dispersion in said protective agent.

4. The unit dosage form according to claim 1, wherein said active ingredient is coated with said protective agent.

5. The unit dosage form according to claim 1, wherein said protective agent is a cationic polymethacrylate.

6. The unit dosage form according to claim 5, wherein said cationic polymethacrylate is a copolymer based on di-C₁₋₄-alkyl-amino-C₁₋₄-alkyl methacrylates and neutral methacrylic acid C₁₋₆-alkyl esters.

7. The unit dosage form according to claim 5, wherein said cationic polymethacrylate is a copolymer based on dimethylaminoethyl methacrylate and neutral methacrylic acid C₁₋₄-alkyl esters.

8. The unit dosage form according to claim 5, wherein said cationic polymethacrylate is a copolymer based on dimethyl-aminoethyl methacrylate, methacrylic acid methyl ester and methacrylic acid butyl ester.

9. The unit dosage form according to claim 5, wherein said cationic polymethacrylate is poly(butyl methacrylate, (2-dimethyl aminoethyl) methacrylate, methyl methacrylate) 1:2:1.

10. The unit dosage form according to claim 1, wherein said protective agent is a wax.

11. The unit dosage form according to claim 10, wherein said wax is carnauba wax.

12. The unit dosage form according to claim 1, wherein said water-soluble matrix polymer is a cellulosic material, a gum, a protein, a starch, a synthetic polymer, a glucan, or a mixture thereof.

13. (canceled)

14. The unit dosage form according to claim 12, wherein said cellulosic material is carboxymethyl cellulose, methyl cellulose, ethyl cellulose, hydroxymethyl cellulose, hydroxyethyl cellulose, hydroxypropyl cellulose, hydroxymethylpropyl cellulose or hydroxypropylmethyl cellulose.

15. The unit dosage form according to claim 13, wherein said cellulosic material is hydroxypropylmethyl cellulose or hydroxypropyl cellulose.

16. (canceled)

17. The unit dosage form according to claim 12, wherein said synthetic polymer is a polyvinyl alcohol polyethylene glycol (PVA-PEG) copolymer.

18. The unit dosage form according to claim 1, wherein said film matrix has a thickness of ≦250 μm, ≦200 μm, ≦150 μm, ≦120, or ≦100 μm.

19. The unit dosage form according to claim 1, wherein said film matrix has a thickness in the range of from 10-150 μm, 20-125 μm, 30-100 μm, 35-90 μm, or 40-80 μm.

20. (canceled)

21. The unit dosage form according to claim 1, wherein said unit dosage form further comprises at least one surfactant.

22. The unit dosage form according to claim 1, wherein said film matrix further comprises at least one surfactant.

23. The unit dosage form according to claim 1, wherein less than 25% (w/w), less than 20% (w/w), less than 15% (w/w), or less than 5% (w/w) of the active ingredient is dissolved from the unit dosage form within 3 minutes when the unit dosage form is placed into a beaker with 10 ml of simulated saliva pH 6.0 at 37° C. as dissolution medium.

24. (canceled)
